(19) United States
(12) Patent Application Publication
(10) Pub. No.: US 2024/0068998 A1
(43) Pub. Date: Feb. 29, 2024

(54) Gas Chromatography Thermal Conductivity Detector (TCD) Calibration Valve

(71) Applicant: OHIO STATE INNOVATION FOUNDATION, Columbus, OH (US)

(72) Inventors: Jacob WILDOFSKY, Columbus, OH (US); Kenneth COPLEY, Columbus, OH (US); Boian ALEXANDROV, Columbus, OH (US)

(21) Appl. No.: 18/239,049

(22) Filed: Aug. 28, 2023

Related U.S. Application Data
(60) Provisional application No. 63/401,398, filed on Aug. 26, 2022.

(51) Int. Cl. G01N 30/86; G01N 30/66; G01N 33/00

(52) U.S. Cl. CPC G01N 30/8665; G01N 30/66; G01N 33/005; G01N 2030/025

(57) ABSTRACT

Provided herein are calibration valves that can be used to direct the flow of gases within a gas chromatograph (GC) equipped with a thermal conductivity detector (TCD). The calibration valves can provide both gas calibration and flow control to a GC TCD in a single compact design. For example, the calibration valves can serially deliver a series of small, predetermined volumes of a gas of interest to a GC TCD to facilitate calibration of the GC TCD for detection and/or quantification of the gas of interest in a sample. In certain aspects, the calibration valve can be designed to calibrate the GC TCD with small volumes of a gas of interest (e.g., hydrogen) ranging from 5 mm³ to 100 mm³ with high precision. Such a calibration valve can be used to calibrate a GC TCD to detect and/or quantify the gas of interest in a sample.

### CROSS-REFERENCE TO RELATED APPLICATIONS

This application claims benefit of U.S. Provisional Application No. 63/401,398, filed Aug. 26, 2022, which is hereby incorporated herein by reference in its entirety.

### BACKGROUND

Gas chromatography is a technique which is used to separate and detect the components of a mixture of gases. Gas chromatography is typically carried out using a separation column. The gas mixture to be separated into its constituent components is carried through the column using a carrier gas (‘mobile phase’). The column is provided with a stationary phase (e.g., a coating on an inner surface of the column). The stationary phase retards the different components of the gas mixture to different extents, in a conventionally-known manner. As a result, the different components are passed through the separation column at different rates, and elute from the column at different, characteristic times, known as retention times.

As the separated components elute from the column, a detector presented to the gas flow detects the components eluted from the column. Suitable detectors are known which detect the thermal conductivity of a gaseous environment. These are referred to in the art as thermal conductivity detectors (TCDs). These rely on the fact that different gas components have different thermal conductivities, and in particular, a different thermal conductivity from the mobile phase. As one component reaches the detector, the change in thermal conductivity of the gaseous environment registers as a peak. The change in thermal conductivity of the gaseous environment, along with the retention time, can then be used to identify the component.

One benefit of TCDs is that they do not rely on any kind of chemical reaction. They are able to detect not only the presence of the different components in the environment, but given the identity of the expected components, they can also provide information related to the concentration of the components.

The basic operating principle of a typical known TCD is to have a heated filament located to be in thermal contact with a gaseous analyte. A change in composition of the analyte typically changes the thermal conductivity of the analyte. Therefore, the rate at which the heated filament loses heat to the analyte also changes, resulting in a change in temperature of the heated filament. This change in temperature is usually measured as a change in electrical resistance of the heated filament. A well-known example of a device which relies on thermal conductivity to measure low gas pressures is a Pirani gauge, which has a heated filament exposed to the gas. The lower the pressure, the lower the rate of heat loss to the surroundings. Therefore, by measuring the temperature of the filament, one can infer the gas pressure (i.e., the extent of the vacuum).

As mentioned above, TCDs can be used in gas chromatography, but can also be used in sensors for any mixture of gases, for example hydrogen and natural gas, or air and fuel in combustion systems. However, to accurately detect and/or quantify a gas of interest, the TCD must be calibrated with the gas of interest. Accordingly, devices, systems, and methods for accurately

### SUMMARY

Provided herein are calibration valves that can be used to direct the flow of gases within a gas chromatograph (GC) equipped with a thermal conductivity detector (TCD). The calibration valves can provide both gas calibration and flow control to a GC TCD in a single compact design. For example, the calibration valves can serially deliver a series of small, predetermined volumes of a gas of interest (referred to herein as a measured gas) to a GC TCD to facilitate calibration of the GC TCD for detection and/or quantification of the gas of interest in a sample. In certain aspects, the calibration valve can be designed to calibrate the GC TCD with small volumes of a gas of interest (e.g., hydrogen) ranging from 5 mm³ to 100 mm³ with high precision. Such a calibration valve can be used to calibrate a GC TCD to detect and/or quantify the gas of interest in a sample having a volume in this range. Of course, if desired for a particular application, the calibration valve can be reasidly modified to increase and/or decrease the volumes of the gas of interest delivered to the GC TCD for calibration (e.g., by altering the volume, for example the depth, of the internal channels present in the disk member, as discussed in more detail below).

For example, in some aspects, described herein are calibration valves that include a rotatable volumes disk member including a plurality of internal channels formed therewithin, wherein each of the internal channels is dimensioned to house a different predetermined volume of a measured gas; a static sealing disk member compressed against the volumes disk member creating a gas seal and allowing for frictionless rotation there between; a static base member including inlet ports and outlet ports for receiving compression fittings to fluidly connect the calibration valve to a gas chromatography system; and an alignment ring member affixed to the base to generate a compressive force on the sealing disk member.

In some aspects, the inlet ports and and outlet ports can include an inlet port and an outlet port fluidly connectable to a carrier gas, an inlet port and an outlet port fluidly connectable to a measured gas used for calibration, and an inlet port and an outlet port fluidly connectable to a sample chamber.

In some aspects, the calibration valve can further include fitting members disposed between the sealing disk member and the base member, and wherein the fitting members house inlet and outlet tubes to prevent gas leakage when the calibration valve is in operation. In some aspects, the fitting members include a conical fitting formed at least in part from polytetrafluoroethylene (PTFE).

In some aspects, the the rotatable volumes disk member is formed at least in part from stainless steel. In some aspects, the internal channels of the volumes disk member are machined. In some aspects, each of the internal channels is dimensioned to house a different predetermined volume of a measured gas that range from a first volume less than a sample's expected measured gas content to a second volume greater than the sample's expected measured gas content. In certain aspects, each of the internal channels has a volume of from 5 mm³ to 100 mm³. In some aspects, the volumes disk member includes at least six internal channels, such as from eight internal channels to twelve internal channels.

In some aspects, the the alignment ring member generates an audible click when the volumes disk member is rotated into a designated position.

In some aspects, the internal channels of the volumes disk member are configured to rotatably align with the base member in multiple set positions including a sample test position, an off position, and a plurality of volume test positions. In some aspects, when the calibration valve is fluidly connected to a gas chromatography system and when the volumes disk member is in the sample test position, a carrier gas flows through a sample chamber to a thermal conductivity sensor of the gas chromatography system. In some aspects, when the calibration valve is fluidly connected to a gas chromatography system and when the volumes disk member is in the off position, no gas flows to the gas chromatography system. In some aspects, when the calibration valve is fluidly connected to a gas chromatography system and when the volumes disk member is in one of the volume test positions, a predetermined volume of a measured gas contained within one of the internal channels formed within the volumes disk member flows to the gas chromatography system for calibration.

In some aspects, the calibration valve can further including a selector, such as a handle or knob, coupled to the volumes disk member and configured to rotatably align the volumes disk member with the base member.

In some aspects, the alignment ring member is affixed to the base via long nose plungers screwed on to the base to generate the compressive force on the sealing disk member. In some aspects, the sealing disk member is formed at least in part from polytetrafluoroethylene (PTFE).

In some aspects, the calibration valve further includes a mount coupled to the base member.

In some aspects, the the volumes disk member and the sealing disk member are disposed between the base member and the alignment ring member.

Also provided herein are systems for the detection or quantification of a measured gas. For example, in some aspects, described herein are systems that include a gas chromatograph equipped with a thermal conductivity detector; and a calibration valve operably coupled to the gas chromatograph, the calibration valve including: a rotatable volumes disk member including a plurality of internal channels formed therewithin, wherein each of the internal channels is dimensioned to house a different predetermined volume of a measured gas; a static sealing disk member compressed against the volumes disk member creating a gas seal and allowing for frictionless rotation there between; a static base member including inlet ports and outlet ports for receiving compression fittings to fluidly connect the calibration valve to a gas chromatography system; and an alignment ring member affixed to the base to generate a compressive force on the sealing disk member; wherein the base member includes an inlet port and an outlet port fluidly connected to a carrier gas, an inlet port and an outlet port fluidly connected to a measured gas used for calibration, and an inlet port and an outlet port fluidly connected to a sample chamber of the gas chromatograph.

Also provided herein are methods for detecting and/or quantifying a measured gas in a sample. For example, in some aspects, provided herein are methods that include providing a gas chromatograph equipped with a thermal conductivity detector; calibrating the gas chromatograph equipped with the thermal conductivity detector for the measured gas using a calibration valve operably coupled to the gas chromatograph, the calibration valve including: a rotatable volumes disk member comprising a plurality of internal channels formed therewithin, wherein each of the internal channels is dimensioned to house a different predetermined volume of a measured gas; a static sealing disk member compressed against the volumes disk member creating a gas seal and allowing for frictionless rotation there between; a static base member comprising inlet ports and outlet ports for receiving compression fittings to fluidly connect the calibration valve to a gas chromatography system; and an alignment ring member affixed to the base to generate a compressive force on the sealing disk member; wherein the base member includes an inlet port and an outlet port fluidly connected to a carrier gas, an inlet port and an outlet port fluidly connected to a measured gas used for calibration, and an inlet port and an outlet port fluidly connected to a sample chamber of the gas chromatograph; and passing the sample comprising the measured gas through the gas chromatograph to detect and/or quantify the measured gas in the sample.

In some aspects, the measured gas includes hydrogen. In certain aspects, the calibrating step facilitates the accurate detection of a volume of diffused hydrogen and the diffusion rate of small weld metal samples within a 5 mm³ to 100 mm³ range, following ISO standard 3690 for hydrogen measurement.

### DESCRIPTION OF DRAWINGS

FIG. 1 depicts a gas chromatograph (GC) equipped with a thermal conductivity detector (TCD).

FIG. 2 is a schematic depiction of the gas flow within an example gas GC equipped with a TCD and a calibration valve described herein. 1 represents a flow rate regulator, 2 represents a control valve, 3 represents calibrating volumes, 4 represents a thermostat, 5 represents a TCD, 6 represent flow rate meters, 7 represents a desorption chamber, 8 represents a specimen, and 9 represents an oven.

FIG. 3 is a schematic illustration of an example Wheatstone bridge circuit for use in a TCD.

FIG. 4 is a plot showing example calibration voltage signal results obtained using a calibration valve described herein.

FIG. 5 illustrates an example of a software interface used as part of a hydrogen measurement system that employs a GC equipped with a TCD and a calibration valve described herein.

FIG. 6 shows an example calibration curve (including a calibration curve equation) for hydrogen gas using a generated a GC equipped with a TCD and a calibration valve described herein.

FIGS. 7A and 7B shows an exploded view of an example calibration valve described herein.

FIG. 8 shows schematic drawings of an example base member.

FIG. 9 shows schematic drawings of an example mount for an example calibration valve described herein.

FIG. 10 shows schematic drawings of an example alignment ring member.

FIG. 11 shows schematic drawings of an example volumes disk member.

FIG. 12 shows schematic drawings of an example sealing disk member.

FIG. 13 shows schematic drawings for example fitting members.

FIG. 14 shows an exploded view of an example calibration valve described herein from the rear.

FIG. 15 schematically illustrates how the base member and the alignment ring member assemble in the example calibration valve described herein.

FIG. 16 schematically illustrates how the base member, volumes disk member, and sealing disk member assemble in the example calibration valve described herein.

FIG. 17 is a calibration valve position guide.

FIG. 18 is a photograph showing a DHCT test stand.

FIG. 19 is a photograph showing a DHCT sample.

FIG. 20 shows a sample test calibration curve prepared using a conventional calibration valve.

FIG. 21 is a photograph showing a hydrogen charging booth.

FIG. 22 is a photograph showing an F22 steel test sample.

FIG. 23 is a plot showing the hydrogen diffusion rate in an F22 steel test sample.

FIG. 24 is a plot showing the hydrogen content (in ppm) of an F22 steel test sample as a function of charging time.

FIG. 25 is a plot showing a 48-hour calibration curve.

FIG. 26 is a plot showing a 48-hour calibration volume peaks.

FIG. 27 is a plot showing an accuracy test calibration curve generated using an example calibration curve described herein.

### DETAILED DESCRIPTION

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

The term “about,” as used herein, refers to variation in the numerical quantity that can occur, for example, through typical measuring techniques and equipment, with respect to any quantifiable variable, including, but not limited to mass, volume, time, temperature, distance, molecular weight, and water permeability. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The embodiments of this invention are not limited to particular devices or methods described herein, which can vary greatly without departing from the scope of the invention.

At the moment, there are relatively few gas chromatography (GC) and other gas measurement devices that are capable of accurately measuring hydrogen gas available in the market. However, there is a significant need for such systems. For example, companies that manufacture products containing welds as well as distributors of the welding equipment need systems and methods for characterizing the strength and susceptibility of their products to hydrogen assisted cracking. In addition, most current commercially available GC systems are designed for measuring volumes of hydrogen above 100 mm³. Their high prices limit their use for smaller applications.

Thermal conductivity detectors are currently the most accurate commercially available detectors for measuring hydrogen gas content. A calibration valve that can be interfaced with conventional GC machines and facilitate calibration and measurement capabilities for gases of interest, including hydrogen, to these systems is of significant importance. Such calibration valves could be attached to commercial GC instruments to provide gas (e.g., hydrogen) calibration and measurement capabilities to the system. This will allow companies who currently own a conventional gas measurement system (e.g., a conventional GC) to acquire improved gas measurement capabilities (e.g., hydrogen measurement capabilities) without purchasing an entirely different system at a higher cost.

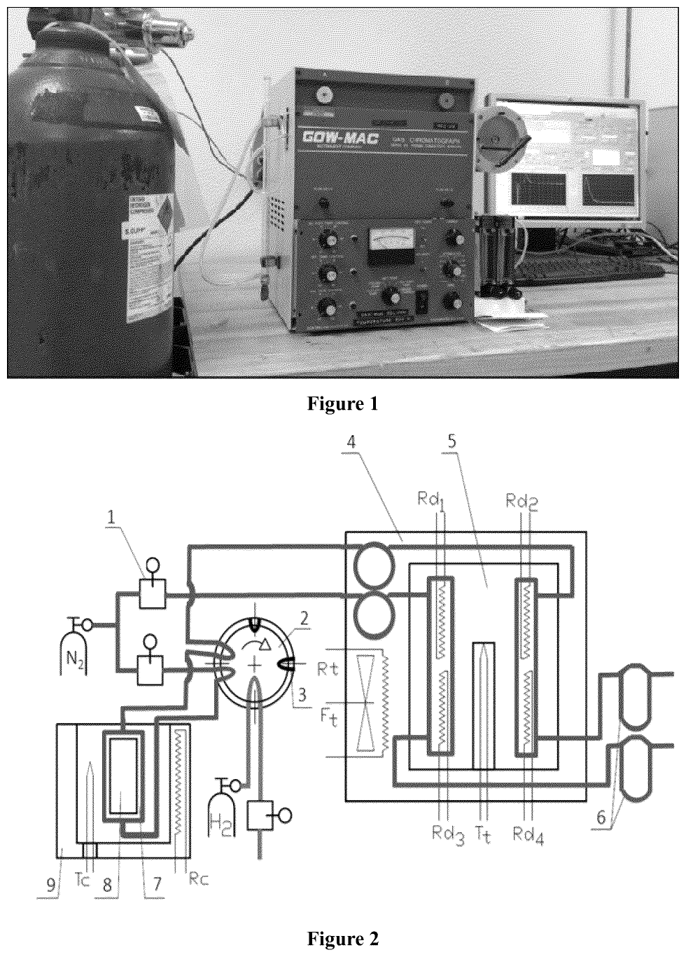

Accordingly, described herein are calibration valves that can be interfaced with a conventional GC thermal conductivity detector (TCD). See FIGS. 1 and 2. The calibration valves described herein can be used to calibrate a TCD to facilitate the accurate detection of a measured gas (e.g., hydrogen) by the TCD. In certain embodiments, the calibration valves described herein can be used to calibrate a TCD to facilitate the accurate detection of a volume of diffused hydrogen and the diffusion rate of small weld metal samples within a 5-100 mm³ range, following ISO standard 3690 for hydrogen measurement.

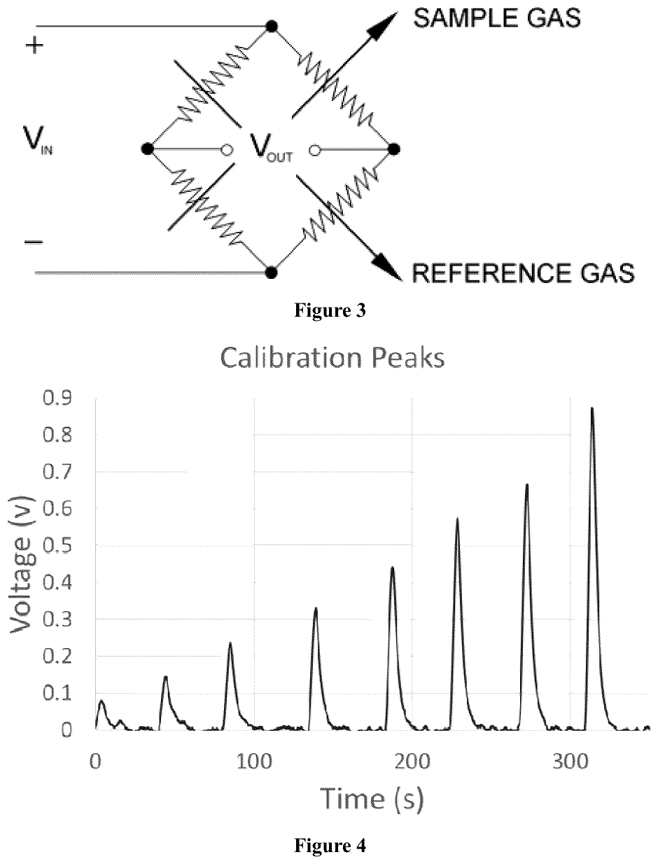

A GC TCD apparatus utilizes two gas lines: one flows a chosen carrier gas (nitrogen) through it and the other flows a mixture of the carrier gas and a chosen measured gas (e.g., hydrogen). These two gas lines pass through the TCD, which contains a Wheatstone bridge circuit of four heated resistors (FIG. 3). Every gas possesses a different coefficient of thermal conductivity. Therefore, when different gases pass over the TCD resistors, they proportionally affect the resistors' temperatures and change their relative resistance. By way of example, nitrogen has a thermal conductivity of 5.680 and hydrogen has a conductivity of 39.60 (Table 1). As a consequence, in a system in which nitrogen serves as the carrier gas and hydrogen is the measured gas, the gas line containing a mixture of nitrogen and hydrogen will pull more heat away from its resistors than those in contact with the nitrogen gas line. The Wheatstone bridge converts the resulting differences in resistance into a voltage signal output proportional to the difference in resistor temperatures. This voltage signal is linearly proportional to the volume of the measured gas present in the mixture of the carrier gas and measured gas, providing a ready means for calculating the concentration of a measured gas in a sample.

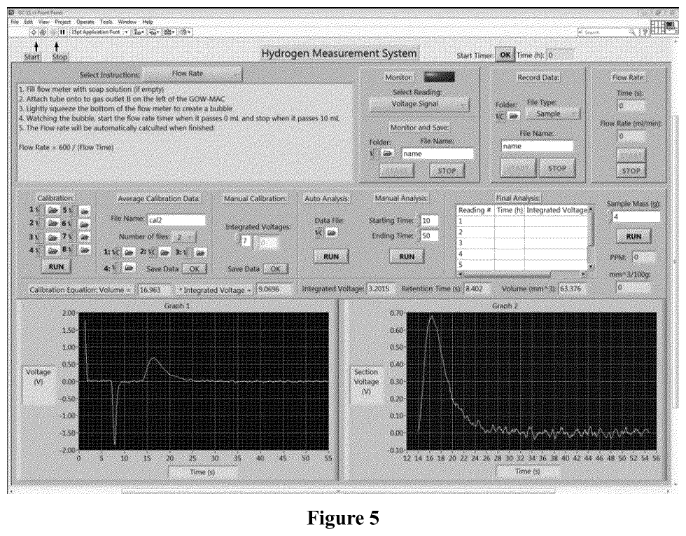

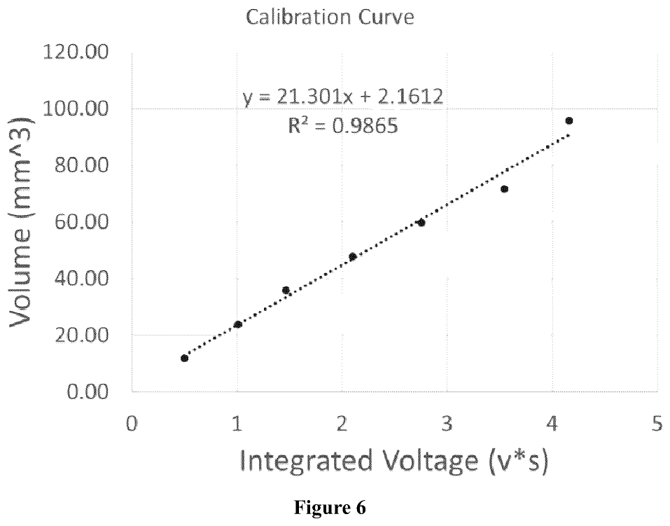

The calibration valves described herein can serially deliver a plurality of exact volumes of a measured gas through the GC TCD for calibration. In certain embodiments, the calibration valves described herein can serially deliver a plurality of volumes of a measured gas (e.g., hydrogen) ranging from 5-100 mm³ through the GC TCD for calibration (FIG. 4). The GC TCD can then record the voltage signals created by these measured gas volumes to create a calibration equation to relate the integrated voltage to the volume of measured gas (FIG. 5). This equation can allow the system to calculate a volume of a measured gas in a test sample as a function of the voltage signal (FIG. 6). If desired, the diffusion rate of the sample can then be found by calculating the diffused gas content over multiple intervals of time. In this way, the the GC TCD to then be used for the accurate detection of a measured gas in a sample.

TABLE 1
Thermal conductivity of some common gases.

| GAS | THERMAL CONDUCTIVITY |
|---|---|
| ACETYLENE | 4.400 |
| AMMONIA | 5.135 |
| ARGON | 3.880 |
| CARBON DIOXIDE | 3.393 |
| CARBON MONOXIDE | 5.425 |
| CHLORINE | 1.829 |
| ETHANE | 4.303 |
| ETHYLENE | 4.020 |
| HELIUM | 33.60 |
| HYDROGEN | 39.60 |
| HYDROGEN SULPHIDE | 3.045 |
| METHANE | 7.200 |
| NEON | 10.87 |
| NITRIC OXIDE | 5.550 |
| NITROGEN | 5.680 |
| NITROUS OXIDE | 3.515 |
| OXYGEN | 5.700 |
| SULPHUR DIOXIDE | 1.950 |

In some embodiments, the calibration valve descrived herein can be distinct from conventional calibration valves because it can fill and send exact volumes of a measured gas through the GC TCD using internal channels that are sealed from leakage, for example, following the ISO standard 3690 for hydrogen measurement. In some embodiments, the calibration valve can include integrated flow controls that allow a user to shut off all air-flow through the GC TCD system, redirect the flow to a sample chamber for testing, and/or send a plurality of predetermined volumes of a measured gas through the GC TCD system for calibration. This allows the user to avoid having to purchase and attach additional solenoid valves for flow control. In this way, the calibration valve can provide an economical and compact solution for users seeking to calibrate conventional GC TCD systems for the detection of a measured gas (e.g., hydrogen).

Calibration Valves

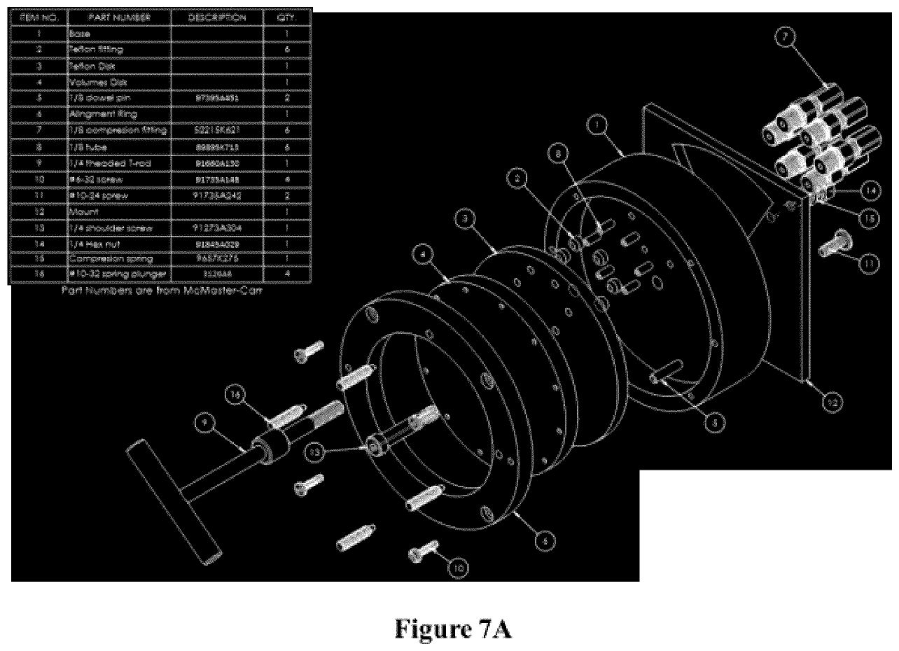

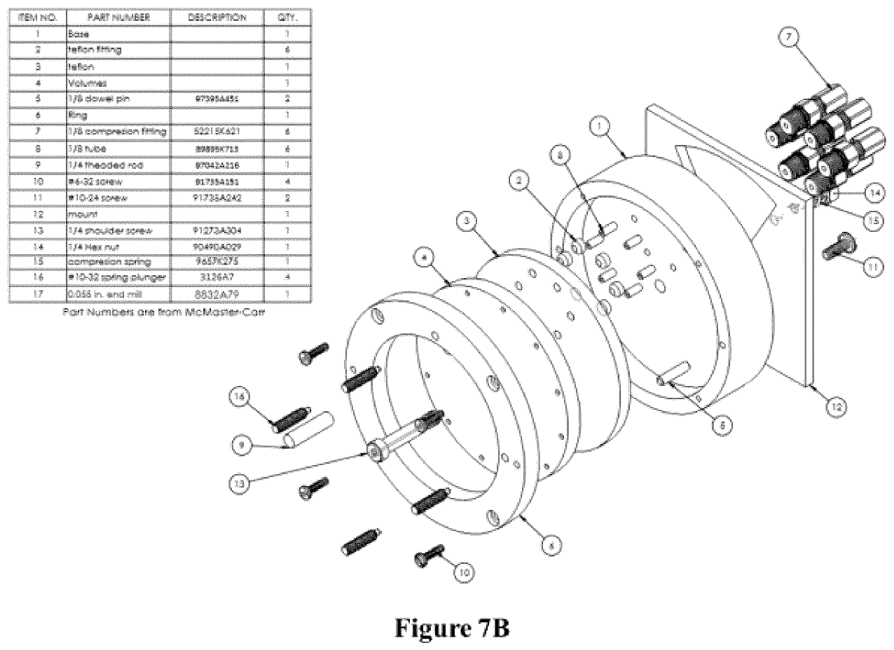

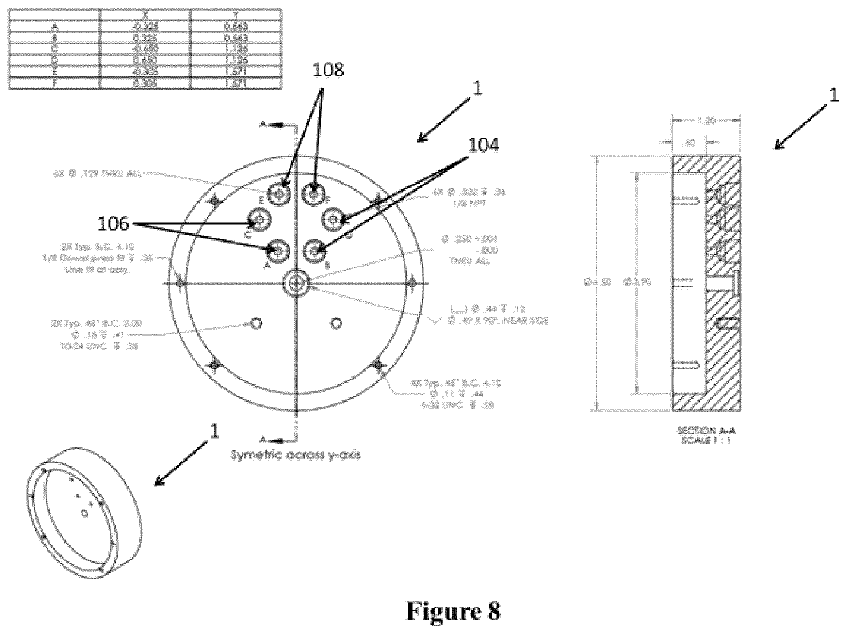

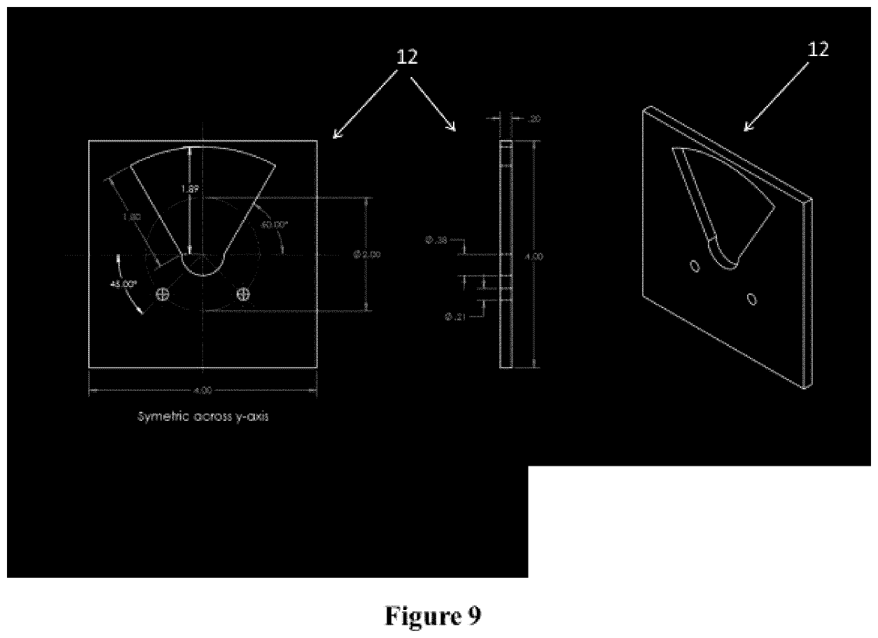

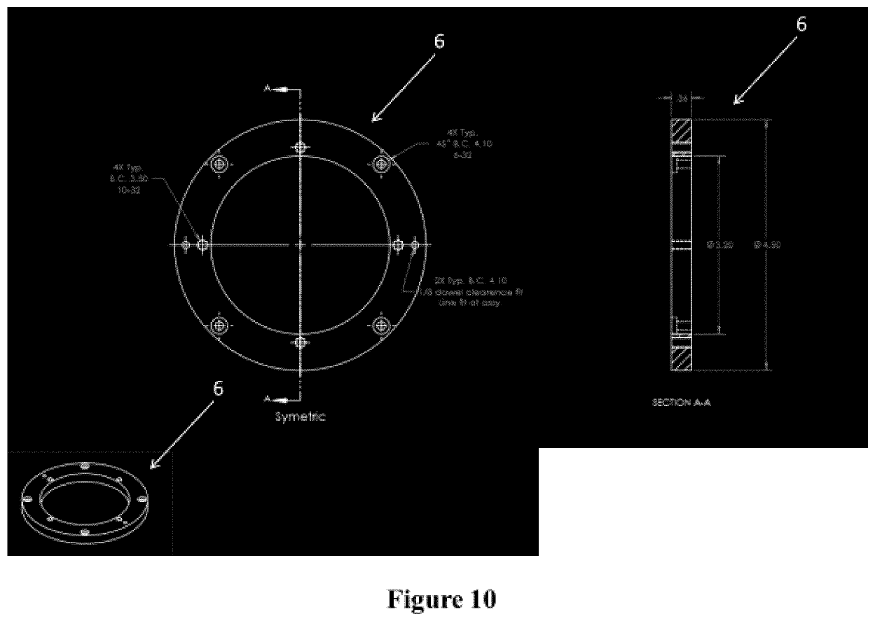

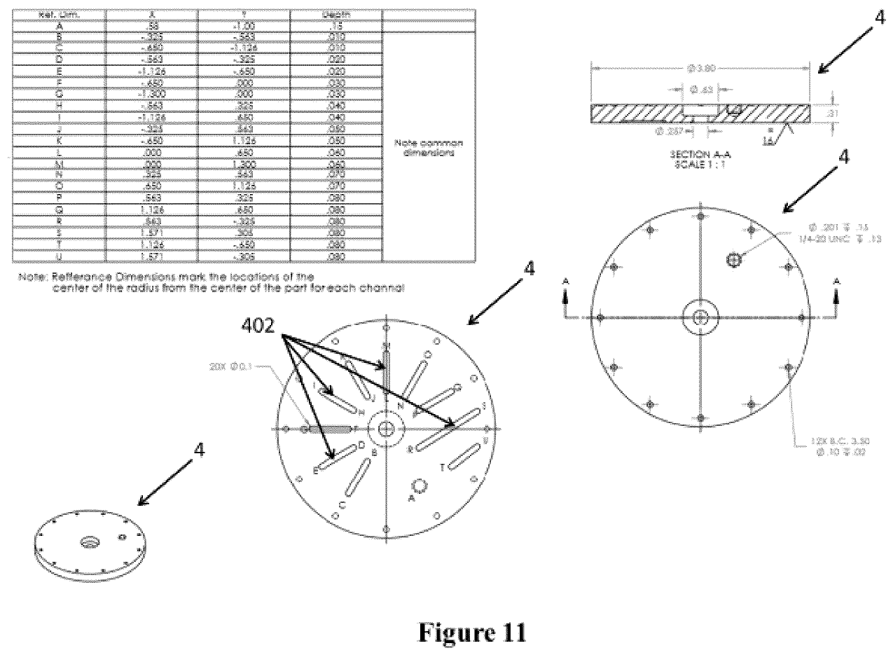

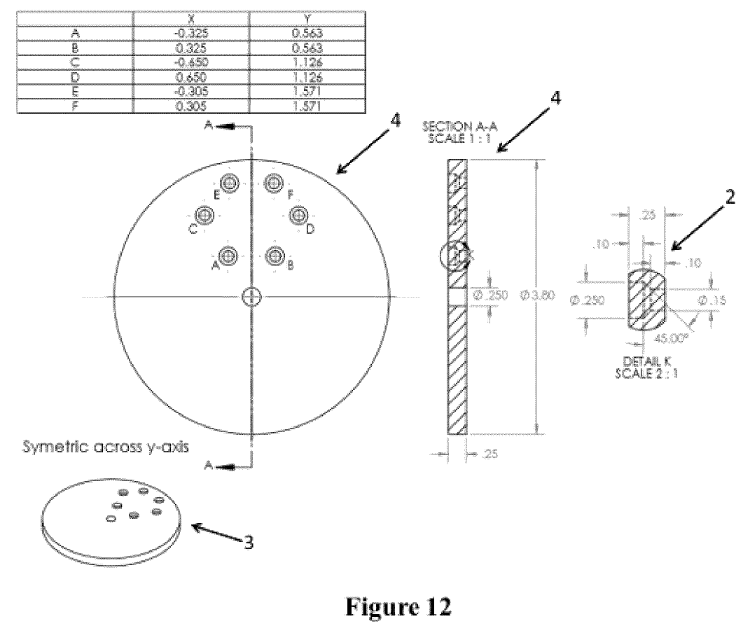

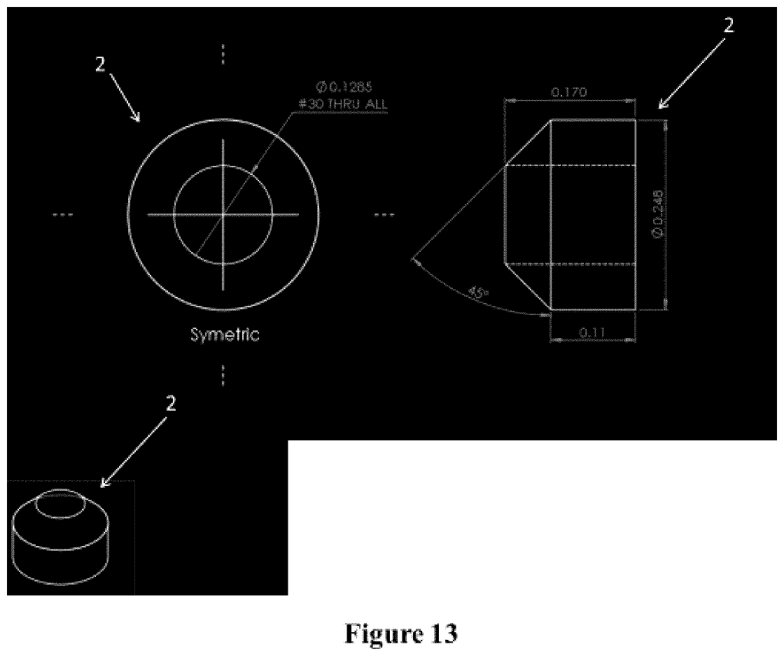

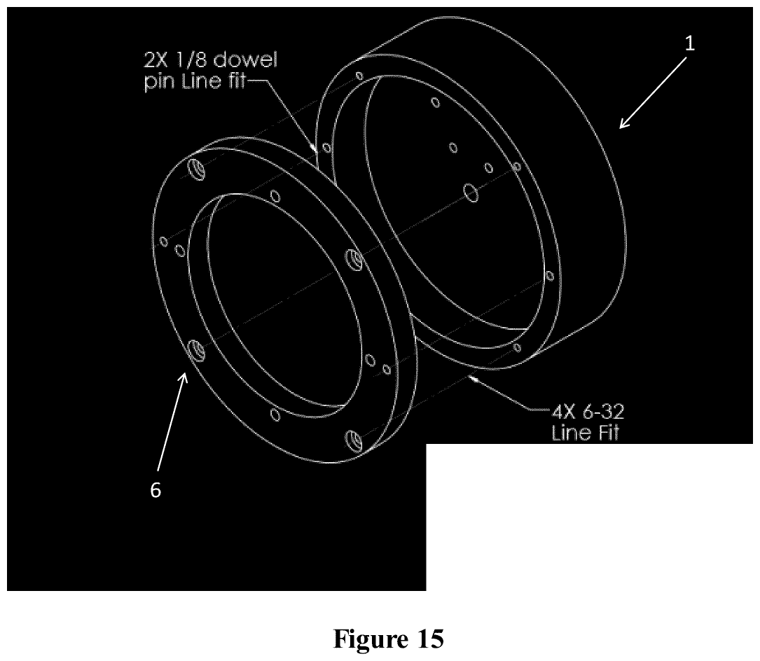

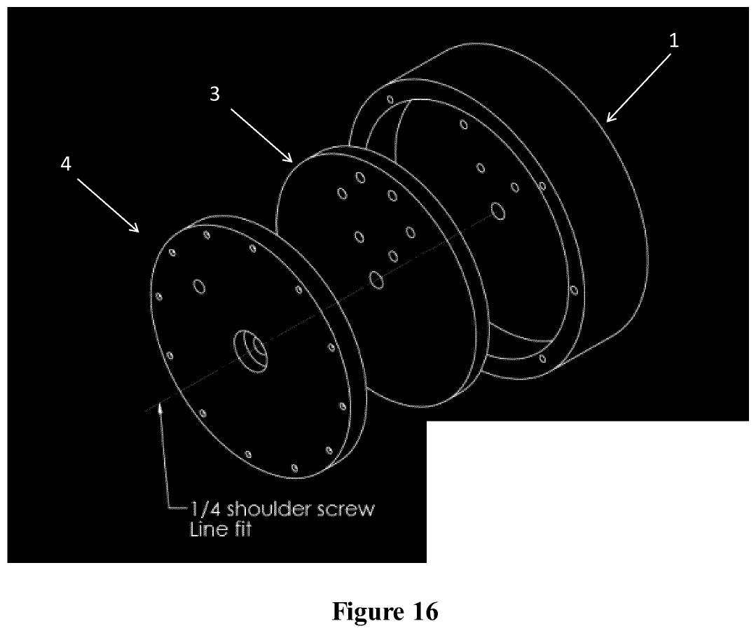

FIGS. 7A-7B show an exploded view of an example calibration valve along with an accompanying parts list. FIGS. 8-16 are the technical drawings for components of the example calibration valve, include a mount (part 12 in FIGS. 7A-7B; further illustrated in FIG. 9), a base member (part 1 in FIGS. 7A-7B; further illustrated in FIG. 8), a sealing disk (part 3 in FIGS. 7A-7B; further illustrated in FIG. 12), fitting members (part 2 in FIGS. 7A-7B; further illustrated in FIG. 13), a volumes disk member (part 4 in FIGS. 7A-7B; further illustrated in FIG. 11), and an alignment ring member (part 6 in FIGS. 7A-7B; further illustrated in FIG. 10).

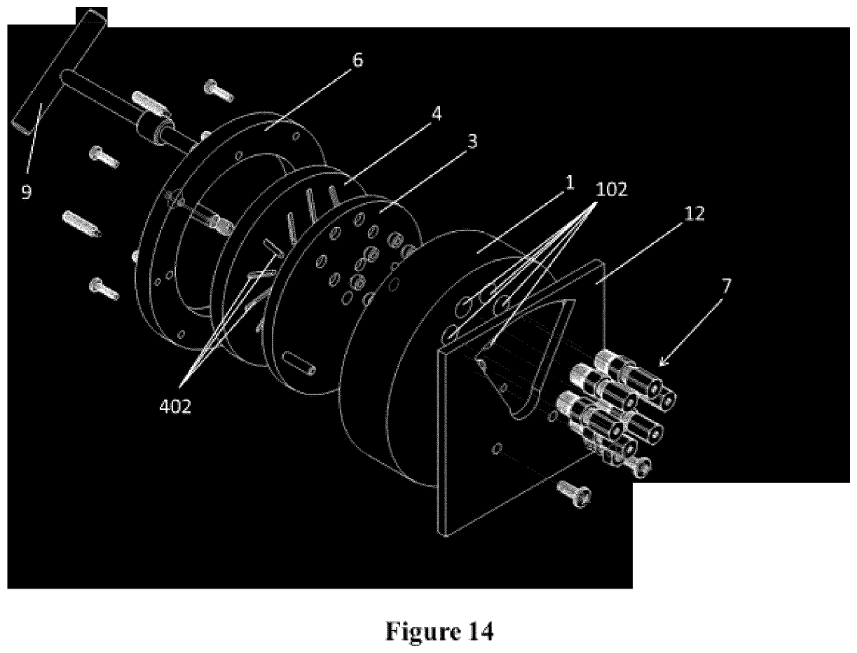

Referring now to FIGS. 7A-7B and FIG. 14, provided herein are calibration valves that include a rotatable volumes disk member (4) including a plurality of internal channels (402) formed therewithin, wherein each of the internal channels is dimensioned to house a different predetermined volume of a measured gas; a static sealing disk member (3) compressed against the volumes disk member creating a gas seal and allowing for frictionless rotation there between; a static base member (1) including inlet ports and outlet ports (102) for receiving compression fittings (7) to fluidly connect the calibration valve to a gas chromatography system; and an alignment ring member (6) affixed to the base (1) to generate a compressive force on the sealing disk member 3).

In some embodiments, the calibration valve further includes a mount (12) coupled to the base member (1). For example, in some embodiments, two screws (part 11, e.g., 10-24 screws) are threaded into the back of the base (1) to attach the valve to the mount (12) and prevent the base (1) from rotating when the volumes disk member (4) is rotated by the user. The mount (see also FIG. 9) as illustrated one includes elements for attaching the valve; however, one of ordinary skill in the art would appreciate that the mount can be extended or otherwise modified to bolt on to any GC TCD system.

In some embodiments, the calibration valve can further include a selector (9), such as a handle or knob, coupled to the volumes disk member (4) and configured to rotatably align the volumes disk member (4) with the base member (1).

In some embodiments, the base member can include three inlet ports and three outlet ports for compression fittings to connect to the inlet and outlet of the carrier gas, the measured gas used for calibration, and a chamber that contains a sample with diffused measured gas. For example, referring now to FIG. 8, the inlet ports and and outlet ports can include an inlet port and an outlet port fluidly connectable to a carrier gas (104), an inlet port and an outlet port fluidly connectable to a measured gas used for calibration (106), and an inlet port and an outlet port fluidly connectable to a sample chamber (108).

In some embodiments, the calibration valve can further include fitting members (2) disposed between the sealing disk member (3) and the base member (1). The fitting members can house inlet and outlet tubes (8, e.g., ⅛ inch stainless steel tubing) to prevent gas leakage when the calibration valve is in operation. In some embodiments, the fitting members can have at least a partially conical shape.

In some embodiments, a pair of rods (5, e.g., two ⅛ inch dowel pins) can extend from the base (1) and sit within clearance holes on the alignment ring member (6) to hold the alignment ring member in position relative to the base. In some embodiments, the alignment ring member (6) can be affixed to the base (1) via long nose plungers (16) screwed on to the base to generate the compressive force on the sealing disk member (3). In some embodiments, the alignment ring member (6) can have threaded holes the long nose plungers (16) that can both compress and align with small indents on the volumes disk member (4), allowing the user to both hear and feel when the volumes disk member (4) enters and/or leaves a designated position when being rotatably aligned with the base member (1). In some embodiments, the the alignment ring member (6) can generate an audible click when the volumes disk member (4) is rotated into a designated position. In some embodiments, one or more additional fasteners can be present to secure the alignment ring member (6) to the base (1). For example, in one embodiments, four additional screws (10, 6-32 screws) can pass through holes in the alignment ring member (6) and thread into the base (1) to supply an even, compressive force to hold the volumes disk member (4) and the sealing disk (part 3) between the base (1) and the alignment ring member (6).

In some embodiments, the internal channels (402) of the volumes disk member (4) are machined. In some embodiments, each of the internal channels is dimensioned to house a different predetermined volume of a measured gas that range from a first volume less than a sample's expected measured gas content to a second volume greater than the sample's expected measured gas content. In certain embodiments, each of the internal channels has a volume of from 5 mm³ to 100 mm³. Example dimensions and volumes for the internal channels shown in the example calibration valve described herein are included in Table 2.

In some embodiments, the volumes disk member can include at least six internal channels (e.g., at least seven internal channels, at least eight internal channels, at least nine internal channels, at least ten internal channels, at least eleven internal channels, at least twelve internal channels, at least thirteen internal channels, at least fourteen internal channels, or at least fifteen internal channels). In some embodiments, the volumes disk member can include sixteen internal channels or less (e.g., fifteen internal channels or less, fourteen internal channels or less, thirteen internal channels or less, twelve internal channels or less, eleven internal channels or less, ten internal channels or less, nine internal channels or less, eight internal channels or less, or seven internal channels or less).

The volumes disk member can include a number of internal channels ranging from any of the minimum values described above to any of the maximum values described above. For example, in some embodiments, the volumes disk member can include from six internal channels to sixteen internal channels (e.g., from eight internal channels to twelve internal channels).

In some embodiments, the internal channels of the volumes disk member are configured to rotatably align with the base member in multiple set positions including a sample test position, an off position, and a plurality of volume test positions. In some embodiments, when the calibration valve is fluidly connected to a gas chromatography system and when the volumes disk member is in the sample test position, a carrier gas flows through a sample chamber to a thermal conductivity sensor of the gas chromatography system. In some embodiments, when the calibration valve is fluidly connected to a gas chromatography system and when the volumes disk member is in the off position, no gas flows to the gas chromatography system. In some embodiments, when the calibration valve is fluidly connected to a gas chromatography system and when the volumes disk member is in one of the volume test positions, a predetermined volume of a measured gas contained within one of the internal channels formed within the volumes disk member flows to the gas chromatography system for calibration.

As discussed above, volumes disk member (4) can include a plurality of internal channels (402) cut to known volumes for calibration and gas flow control. The volumes disk member (402) can have a precision ground flat surface that is gas sealed when compressed against the friction-less sealing disk member's surface. As an additional precaution to seal the disks, conical fittings (2) can be pressed between the sealing disk member (3) and the base (1).

Each part of the calibration valve should be aligned to ensure the valve functions properly. In some embodiments, a shoulder screw (part 13, ¼ inch crew) can pass through the center of the calibration valve to fix the positions of the volumes disk member (4), the sealing disk member (3), and the base member (1) around a common reference center. When paired with a compression spring (15) and a nut (14, ¼ inch hex nut), the shoulder screw can also provide additional compression to the calibration valve.

In the embodiments, the volumes disk member (4) can rotate 360 degrees. For example, in the case of the example calibration valve described herein, the volumes disk member (4) can rotate 360 degrees, allowing the user to select between eleven possible positions (8 calibration volumes, 2 off, and 1 sample test). To actuate this rotation, a selector (9, a ¼ inch T-rod) is threaded into the volumes disk member for the user to turn.

TABLE 2
Example volume disk calibration volumes.

| | Depth of | Area of | Volume of | Volume of |
|---|---|---|---|---|
| | Internal | Internal | Internal | Internal |
| Volume | Channel | Channel | Channel | Channel |
| No. | (in) | (in²) | (in³) | (mm³) |
| 1 | 0.010 | 0.073 | 0.00073 | 11.94 |
| 2 | 0.020 | 0.073 | 0.00146 | 23.88 |
| 3 | 0.030 | 0.073 | 0.0022 | 35.82 |
| 4 | 0.040 | 0.073 | 0.0029 | 47.75 |
| 5 | 0.050 | 0.073 | 0.0036 | 59.69 |
| 6 | 0.060 | 0.073 | 0.0044 | 71.63 |
| 7 | 0.070 | 0.073 | 0.0051 | 83.57 |
| 8 | 0.080 | 0.073 | 0.0058 | 95.51 |
| Short Pass | 0.080 | 0.064 | 0.0051 | 84.07 |
| Long Pass | 0.080 | 0.127 | 0.0101 | 166.11 |

The calibration valves described herein can function by allowing gas to flow through the inlet ports, through the sealing disk member, into and through the internal channels in the volumes disk member, then back through the sealing disk member to the outlet ports (which then flow to the GC equipped with the TCD). Further descriptions of calibration valve positions and calibration valve operation are included below.

In some embodiments, one or more components of the calibration valves described herein can be fabricated, at least in part, from stainless steel. For example, in certain embodiments, the rotatable volumes disk member is formed at least in part from stainless steel. In certain embodiments, the rotatable volumes disk member is formed entirely from stainless steel (e.g., the volumes disk member is machined from stainless steel). In certain embodiments, 304 Stainless steel was chosen for the volumes disk member for its resistance to hydrogen gas diffusion through the material, stiffness and hardness to prevent the internal channels from changing volume, and machinability. However, one of ordinary skill in the art will appreciate that the volumes disk member can be made from other grades of stainless steel and possibly other engineering metals as long as they retain the ability to resist hydrogen gas diffusion through the material (when hydrogen is the measured gas) and maintain constant internal volumes.

In some embodiments, the base, the alignment ring, or a combination thereof can be fabricated (in whole or in part) from aluminum. In some embodiments, both the base and the alignment ring are fabricated (in whole or in part) from aluminum. In certain embodiments, 6061-T6 Aluminum was chosen for its low density to reduce the weight of the valve and machinability. However, one of ordinary skill in the art will appreciate that the base and alignment ring can also be made from other grades of aluminum and possibly other engineering metals as long as they remain suitably stiff to hold the valve in compression.

In some embodiments, the sealing disk member, the fitting members, or a combination thereof are formed at least in part from polytetrafluoroethylene (PTFE, commercially available under the tradename TEFLON®). PTFE was selected to provide a flat, smooth, friction-less surface to allow the volumes disk member to rotate while sealing the valve from leakage.

Systems and Methods of Use

Also provided herein are systems for the detection or quantification of a measured gas. For example, described herein are systems that include a gas chromatograph equipped with a thermal conductivity detector; and a calibration valve operably coupled to the gas chromatograph, the calibration valve including: a rotatable volumes disk member including a plurality of internal channels formed therewithin, wherein each of the internal channels is dimensioned to house a different predetermined volume of a measured gas; a static sealing disk member compressed against the volumes disk member creating a gas seal and allowing for frictionless rotation there between; a static base member including inlet ports and outlet ports for receiving compression fittings to fluidly connect the calibration valve to a gas chromatography system; and an alignment ring member affixed to the base to generate a compressive force on the sealing disk member; wherein the base member includes an inlet port and an outlet port fluidly connected to a carrier gas, an inlet port and an outlet port fluidly connected to a measured gas used for calibration, and an inlet port and an outlet port fluidly connected to a sample chamber of the gas chromatograph.

Also provided herein are methods for detecting and/or quantifying a measured gas in a sample. For example, provided herein are methods that include providing a gas chromatograph equipped with a thermal conductivity detector; calibrating the gas chromatograph equipped with the thermal conductivity detector for the measured gas using a calibration valve described herein; and passing the sample comprising the measured gas through the gas chromatograph to detect and/or quantify the measured gas in the sample.

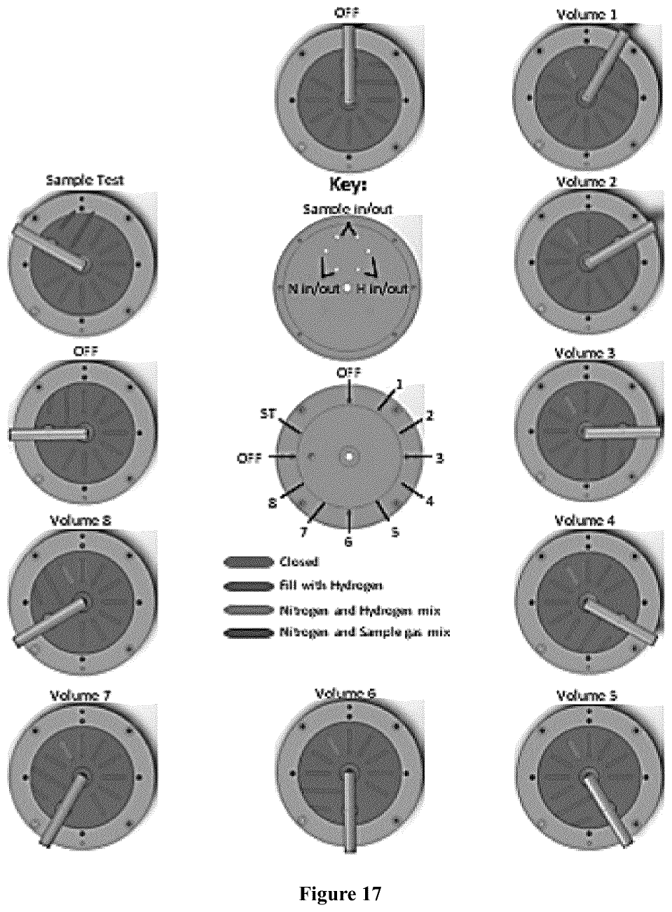

An example operation workflow for calibration valve operation is included below. Reference in the example operation workflow is made to FIG. 17, which illustrates example valve positions for an example calibration valve described herein. By way of example, hydrogen is the measured gas and nitrogen is the carrier gas is the example operation workflow below. However, one of ordinary skill in the art would understand that this workflow can be readily adopted for other measured gases and carrier gases. Likewise, the protocol can be adjusted for alternative calibration valve designs (e.g., calibration valve containing volumes disks that include different numbers of internal channels).

Calibration of GC TCD
1. Turn the calibration valve to the left OFF position.
2. Open the nitrogen gas cylinder and set the pressure to 20 psi.
3. Turn on the Gas Chromatography Thermal Conductivity Detector (GC TCD), turn on the switch to the detector power, and rotate the current dial completely clockwise for a maximum current.
4. Turn the calibration valve to position 8 and set the flow rates of both gas lines A and B to 30 ml/min.
5. To start calibration, turn valve to position ST and slightly open the hydrogen cylinder, set pressure to 15 psi, and reduce flow rate to below 10 ml/min to fill volume 8.
6. Rotate valve to left OFF position to fill volume 7 with hydrogen.
7. Rotate valve to position 8 to test volume 8 and fill volume 6.
8. Repeat this process, going around counter-clockwise to test all volumes from 8 to 1.
9. After testing the last volume, turn the valve to the top OFF position and shut off the hydrogen cylinder.
10. If finished using the system, turn off all power switches on the GC TCD, rotate the current dial completely counter-clockwise to shut off the current, and shut off the nitrogen cylinder.
11. For sample testing, skip step 10 and continue below

Sample Testing
12. If not completed already, perform steps 1-3 in Calibration workflow above to set up the GC TCD system.
13. Turn the valve to position ST and set the flow rates of both gas lines A and B to 30 ml/min.
14. Leaving the valve in position ST, open the sample chamber in the GC TCD, insert a prepared sample into the chamber, and quickly close the chamber. While in position ST, the system purges the chamber with nitrogen.
15. Turn the valve to the left OFF position to stop purging the sample chamber.
16. To test the sample at any given point, turn the valve back to position ST to measure the collective hydrogen diffused into the sample chamber, then turn the valve back to the left OFF position when completed.
17. If you are finished using the system, turn off all power switches on the GC TCD, rotate the current dial completely counter-clockwise to shut off the current, and shut off the nitrogen cylinder.

During the workflow above, when waiting over 30 minutes between tests, shut off the gas cylinders to conserve gas.

In some embodiments, the measured gas includes hydrogen. In some embodiments, the calibration valves described herein can be designed to interface with an existing GC TCD to test small metal and welded samples for hydrogen content and hydrogen diffusion rates. The collected data can be used to assess and/or evaluate hydrogen assisted cracking (HAC) in welds. Informed by these results, research groups can evaluate the performance of various welds and engineer new methods to prevent hydrogen assisted cracking. Companies are interested in a solution to hydrogen assisted cracking to prevent large gas pipelines from fracturing and leaking into the environment and other similar applications. In certain embodiments of the methods described herein, calibration via the calibration valve described herein facilitates the accurate detection of a volume of diffused hydrogen and the diffusion rate of small weld metal samples within a 5 mm³ to 100 mm³ range, following ISO standard 3690 for hydrogen measurement.

Although originally built and designed specifically for hydrogen measurement applications, the valve can be functional in a variety of fields. Any system with limited space, multiple gas flows, and a need for set volumes of gas could take advantage of the compact valve design. Alternative uses include applying the valve to other chromatography devices and in the medical field. The valve does not have to be limited to only gas chromatographs paired with a thermal conductivity detector, but any type testing gas samples.

The valve can also be used for medical applications that require multiple exact dosages of a medication, measuring and transferring substances to another system or to a patient.

Only a few hydrogen measurement devices are commercially available. The calibration valves described herein provide both gas calibration and flow control to a GC TCD system in a single compact design. In some embodiments, the calibration valves described herein are built to calibrate a system with small volumes of a measured gas (e.g., hydrogen), such as volumes ranging from 5 mm³ to 100 mm³ with precision (see Table 2). While in some cases the calibration valves described herein are applicable for testing and measuring hydrogen in small samples within these set ranges, one of ordinary skill in the art will appreciate that these calibration valves could be redesigned to fit both smaller and larger application by adjusting the volume of the internal channels in the volumes disk member (e.g., by altering the depth of the internal channels in the volumes disk member).

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. Aspects of these methods (e.g., reaction times, pH values, types and volumes of 2D sheets used, support membrane used, etc.) can be varied from trial to trial. All steps were performed at room temperature.

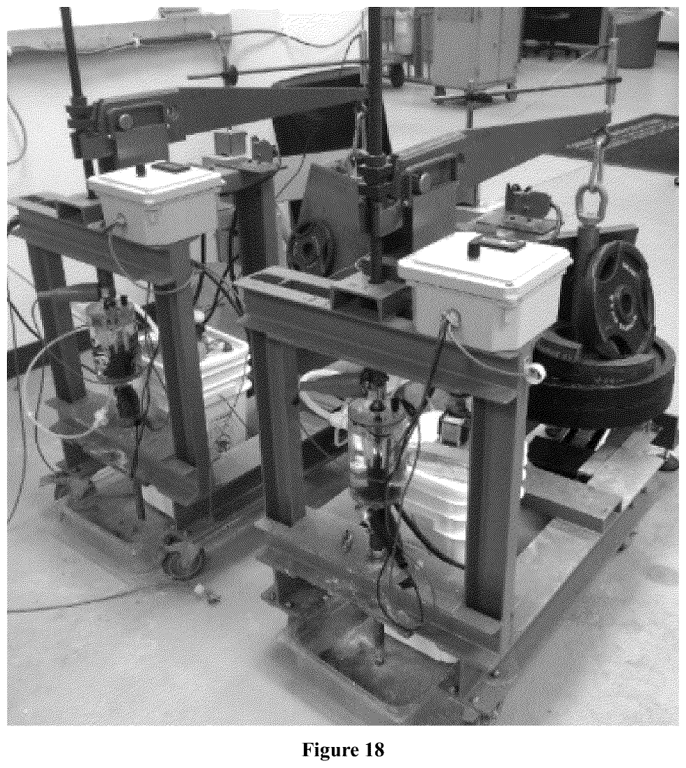

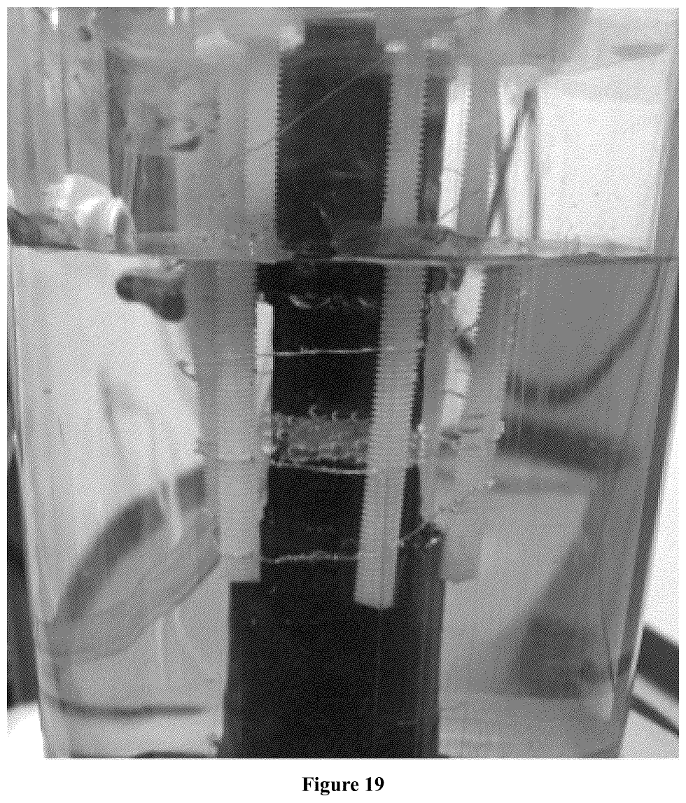

Oil and gas companies have been experiencing catastrophic failures in upstream and downstream structures due to hydrogen assisted cracking (HAC). The cracking originates from dissimilar metal welds (DMWs) on high strength steel components with Ni-base or austenitic stainless steel filler metals after hydrogen slowly diffuses into the dissimilar transition zone in hydrogen containing service environments. The Delayed Hydrogen Cracking Tests (DHCT) can be performed on DMW samples to determine the conditions for failure utilizing constant loading conditions and simultaneous hydrogen charging (FIGS. 18-19). However, improved hydrogen measurement capabilities are needed to support DHCT test characterization. Specifically, a system was needed to quantify the maximum amount of hydrogen which diffuses into the sample before it fails in the DHCT test (hydrogen saturation time) and determine the rate at which hydrogen diffuses in and out of the sample (hydrogen diffusion coefficients).

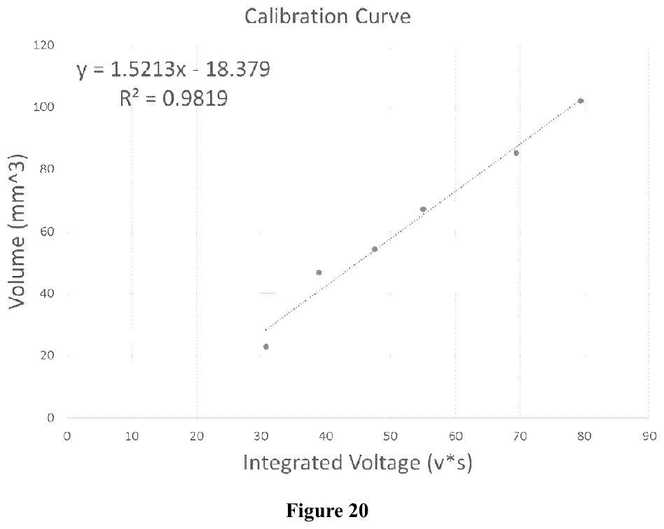

First, data and sample testing was conducted using a conventional calibration valve. FIG. 20 shows a calibration curve created using the conventional calibration valve. Only six volumes were incorporated in the calibration valve Volumes ranged from 20 mm³ to 105 mm³.

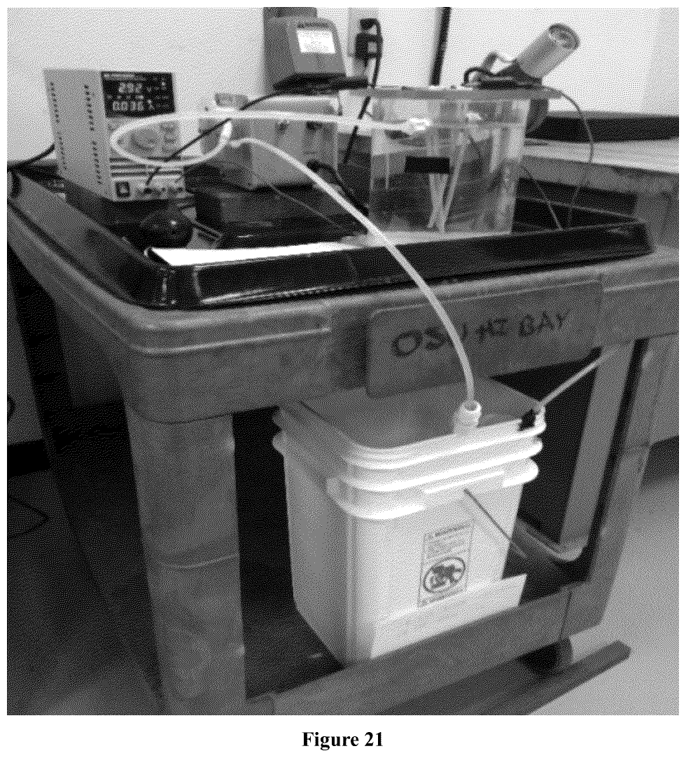

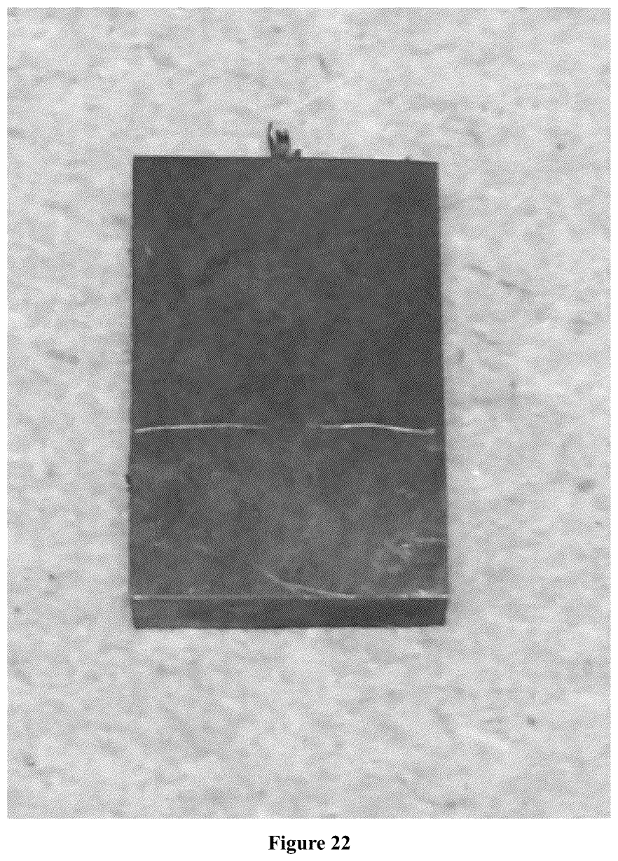

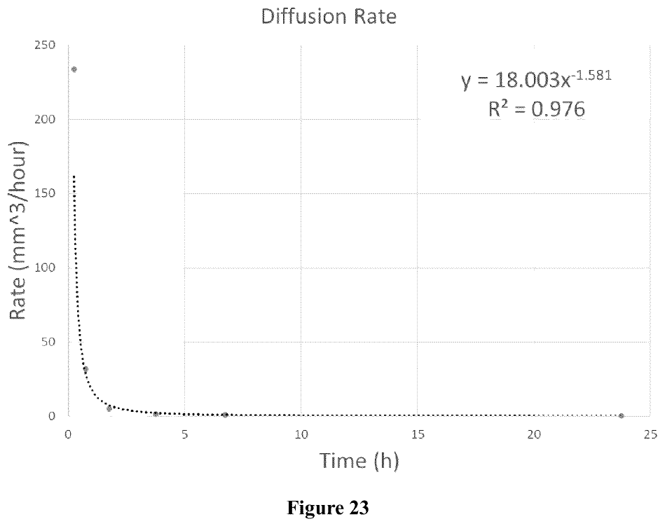

Preliminary tests were performed by charging base metal samples with hydrogen utilizing a charging booth (see FIGS. 21 and 22) similar to the DHCT test, but without a static load. Example sample results shown in Table 3 are from a F22 steel base metal sample with a mass of 2.7663 grams and a volume of 360 mm³. The sample was charged for 90 hours. 87.73 mm³ hydrogen at standard temperature and pressure was detected, resulting in a hydrogen content of 2.82 ppm. The calibration equation produced before the sample test and used for the following calculations was Volume (mm³)=1.672*Integrated Voltage (V*s)−16.92. An estimated diffusion curve shown in FIG. 23 can then be formed from the diffusion rates from each test.

TABLE 3
Results of F22 steel sample test.

| Test | Integrated | Volume | Relative | Total | Difusion Rate |
|---|---|---|---|---|---|
| # | Voltage | (mm\hat{ }3) | Time (h) | time (h) | (mm\hat{ }3/h) |
| 1 | 34.57 | 58.41 | 0.25 | 0.25 | 233.66 |
| 2 | 9.38 | 15.85 | 0.5 | 0.75 | 31.70 |
| 3 | 2.91 | 4.91 | 1 | 1.75 | 4.91 |
| 4 | 1.65 | 2.79 | 2 | 3.75 | 1.39 |
| 5 | 1.41 | 2.38 | 3 | 6.75 | 0.79 |
| 6 | 2.00 | 3.39 | 17 | 23.75 | 0.20 |

Similar to base metal testing, welded samples can be tested for hydrogen content, diffusion rate, and charging saturation time. Results can be used for comparative analysis between samples to determine which welding and treatment processes produce a greater susceptibility to HAC.

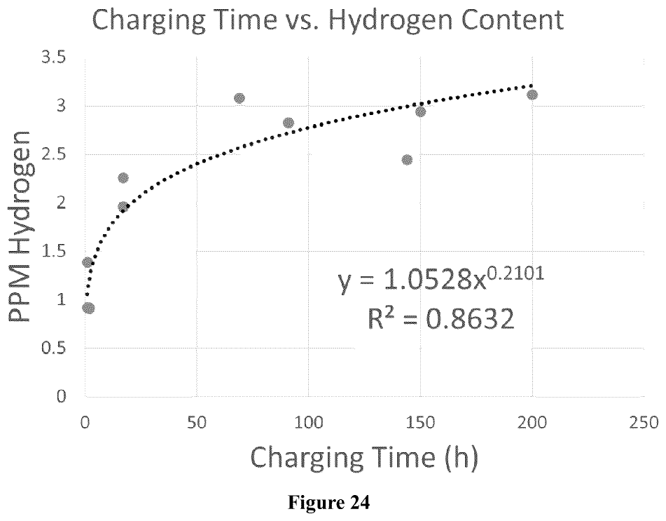

Following the first sample test explained above, multiple tests were completed with charging times ranging from 1 to 200 hours. As a result, a diffusion curve was formed revealing the saturation time for F22 steel to approach maximum hydrogen content around 3 ppm in FIG. 24.

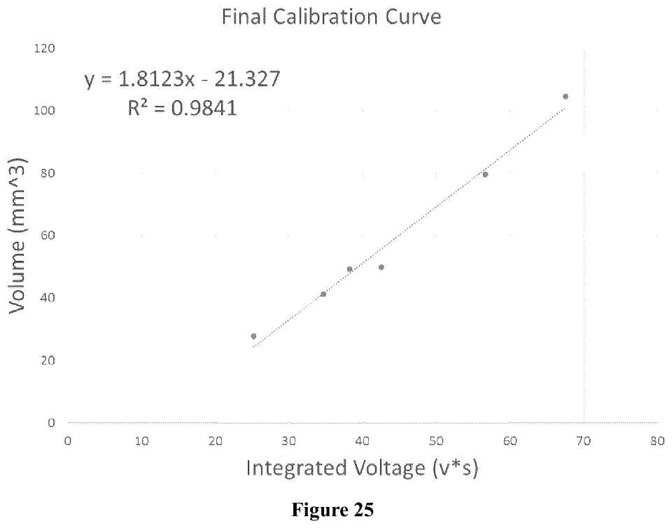

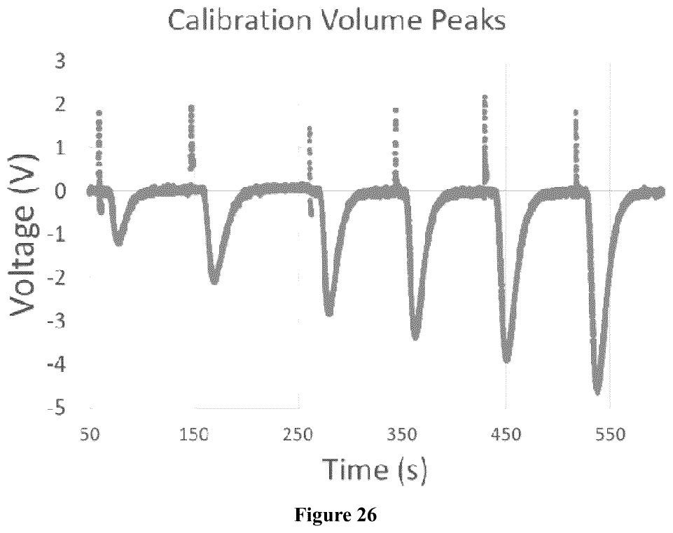

A 48-hour calibration test procedure was designed to characterize the accuracy and repeatability of the conventional calibration valve. The machine was given 4 hours to heat and stabilize before the first calibration, then each calibration was performed 2 hours apart. The machine was left running and recording over night to observe temperature and voltage drift over a longer period of time. For the 10th calibration, the data was recorded 10 times. The results of the test, including sample recorded voltage peaks for each volume, are presented in Table 4 and FIGS. 25 and 26.

TABLE 4
48-hour calibration test results.

| | | Integrated | Standard | |
|---|---|---|---|---|
| | Volume | Voltage | Deviation | equation/error |
| 10th Average | | | | |
| | 27.7392 | 25.12624 | 1.093015375 | Volume = 1.69704*V − 19.5487 |
| | 41.1681 | 37.42623 | 1.765355439 | error= |
| | 49.198 | 39.4814 | 1.444174045 | 0.15848349 |
| | 49.8402 | 44.05354 | 1.578331657 | |
| | 79.5597 | 60.1618 | 2.002838821 | |
| | 104.564 | 70.32246 | 4.331466968 | |
| Total Average | | | | |
| | 27.7392 | 25.196624 | 1.135197553 | Volume = 1.8123*V − 21.327 |
| | 41.1681 | 34.705833 | 1.490020972 | error= |
| | 49.198 | 38.23004 | 1.244299059 | 0.075078449 |
| | 49.8402 | 42.526274 | 1.662616075 | |
| | 79.5597 | 56.65061 | 3.666033889 | |
| | 104.564 | 67.562756 | 6.795271783 | |

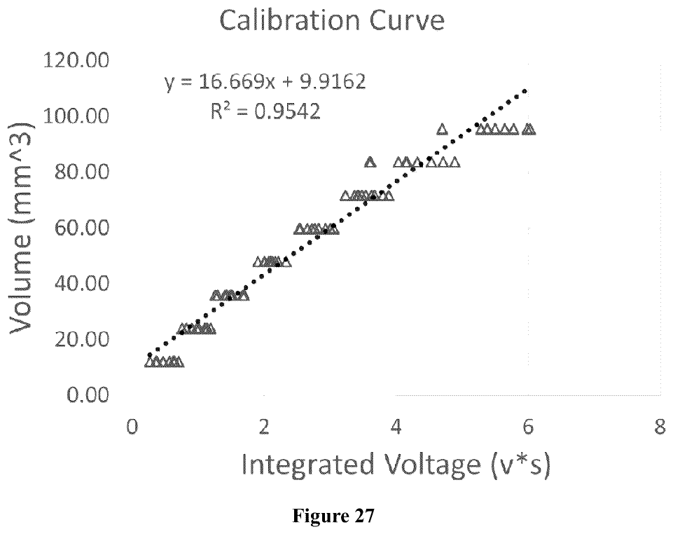

Finally, data was collected using a calibration valve described herein and the results obtained with the calibration valve described herein were compared to those obtained using a conventional calibration valve. Similar to the 48-hour test described herein, an extended calibration test was conducted using a calibration valve described herein, taking voltage readings from each of eight volumes ten times to assess the accuracy and repeatability of the calibration valve described herein. FIGS. 4, 6, and 27 and Table 5 show the results from the tests performed using the calibration valve described herein. This data demonstrates that the calibration valve described herein provides for excellent calibration while following the ISO standard 3690 for hydrogen measurement.

TABLE 5
Summary of accuracy test results.

| Volume | Volume | Average Integrated | Standard |
|---|---|---|---|
| # | (mm\hat{ }3) | Voltage (v*s) | Deviation |
| 1 | 11.94 | 0.50 | 0.15 |
| 2 | 23.88 | 1.01 | 0.15 |
| 3 | 35.82 | 1.47 | 0.14 |
| 4 | 47.75 | 2.10 | 0.12 |
| 5 | 59.69 | 2.76 | 0.19 |
| 6 | 71.63 | 3.54 | 0.20 |
| 7 | 83.57 | 4.16 | 0.47 |
| 8 | 95.51 | 5.52 | 0.41 |

The devices, systems, and methods of the appended claims are not limited in scope by the specific devices, systems, and methods described herein, which are intended as illustrations of a few aspects of the claims. Any devices, systems, and methods that are functionally equivalent are intended to fall within the scope of the claims. Various modifications of the devices, systems, and methods in addition to those shown and described herein are intended to fall within the scope of the appended claims. Further, while only certain representative compounds, components, compositions, elements, and method steps disclosed herein are specifically described, other combinations of the compounds, components, compositions, elements, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A calibration valve comprising:
a rotatable volumes disk member comprising a plurality of internal channels formed therewithin, wherein each of the internal channels is dimensioned to house a different predetermined volume of a measured gas;
a static sealing disk member compressed against the volumes disk member creating a gas seal and allowing for frictionless rotation there between;
a static base member comprising inlet ports and outlet ports for receiving compression fittings to fluidly connect the calibration valve to a gas chromatography system; and
an alignment ring member affixed to the base to generate a compressive force on the sealing disk member.

2. The calibration valve of claim 1, wherein the inlet ports and and outlet ports comprise an inlet port and an outlet port fluidly connectable to a carrier gas, an inlet port and an outlet port fluidly connectable to a measured gas used for calibration, and an inlet port and an outlet port fluidly connectable to a sample chamber.

3. The calibration valve of claim 1, wherein the calibration valve further comprises fitting members disposed between the sealing disk member and the base member, and wherein the fitting members house inlet and outlet tubes to prevent gas leakage when the calibration valve is in operation.

4. The calibration valve of claim 3, wherein the fitting members comprise a conical fitting formed at least in part from polytetrafluoroethylene (PTFE).

5. The calibration valve of claim 1, wherein the rotatable volumes disk member is formed at least in part from stainless steel.

6. The calibration valve of claim 1, wherein the internal channels of the volumes disk member are machined.

7. The calibration valve of claim 1, wherein each of the internal channels is dimensioned to house a different predetermined volume of a measured gas that range from a first volume less than a sample's expected measured gas content to a second volume greater than the sample's expected measured gas content.

8. The calibration valve of claim 1, wherein each of the internal channels has a volume of from 5 mm³ to 100 mm³.

9. The calibration valve of claim 1, wherein the volumes disk member comprises at least six internal channels, such as from eight internal channels to twelve internal channels.

10. The calibration valve of claim 1, wherein the alignment ring member generates an audible click when the volumes disk member is rotated into a designated position.

11. The calibration valve of claim 1, wherein the internal channels of the volumes disk member are configured to rotatably align with the base member in multiple set positions including a sample test position, an off position, and a plurality of volume test positions.

12. The calibration valve of claim 11, wherein when the calibration valve is fluidly connected to a gas chromatography system and when the volumes disk member is in the sample test position, a carrier gas flows through a sample chamber to a thermal conductivity sensor of the gas chromatography system.

13. The calibration valve of claim 11, wherein when the calibration valve is fluidly connected to a gas chromatography system and when the volumes disk member is in the off position, no gas flows to the gas chromatography system.

14. The calibration valve of claim 11, wherein when the calibration valve is fluidly connected to a gas chromatography system and when the volumes disk member is in one of the volume test positions, a predetermined volume of a measured gas contained within one of the internal channels formed within the volumes disk member flows to the gas chromatography system for calibration.

15. The calibration valve of claim 11, further comprising a selector, such as a handle, coupled to the volumes disk member and configured to rotatably align the volumes disk member with the base member.

16. The calibration valve of claim 1, wherein the alignment ring member is affixed to the base via long nose plungers screwed on to the base to generate the compressive force on the sealing disk member.

17. The calibration valve of claim 1, wherein the sealing disk member is formed at least in part from polytetrafluoroethylene (PTFE).

18. The calibration valve of claim 1, further comprising a mount coupled to the base member.

19. The calibration valve of claim 1, wherein the volumes disk member and the sealing disk member are disposed between the base member and the alignment ring member.

20. A system for the detection or quantification of a measured gas, the system comprising:
a gas chromatograph equipped with a thermal conductivity detector; and
a calibration valve operably coupled to the gas chromatograph, the calibration valve comprising:
a rotatable volumes disk member comprising a plurality of internal channels formed therewithin, wherein each of the internal channels is dimensioned to house a different predetermined volume of a measured gas;
a static sealing disk member compressed against the volumes disk member creating a gas seal and allowing for frictionless rotation there between;
a static base member comprising inlet ports and outlet ports for receiving compression fittings to fluidly connect the calibration valve to a gas chromatography system; and
an alignment ring member affixed to the base to generate a compressive force on the sealing disk member;
wherein the base member comprises an inlet port and an outlet port fluidly connected to a carrier gas, an inlet port and an outlet port fluidly connected to a measured gas used for calibration, and an inlet port and an outlet port fluidly connected to a sample chamber of the gas chromatograph.

21. A method for detecting and/or quantifying a measured gas in a sample, the method comprising:
providing a gas chromatograph equipped with a thermal conductivity detector;
calibrating the gas chromatograph equipped with the thermal conductivity detector for the measured gas using a calibration valve operably coupled to the gas chromatograph, the calibration valve comprising:
a rotatable volumes disk member comprising a plurality of internal channels formed therewithin, wherein each of the internal channels is dimensioned to house a different predetermined volume of a measured gas;
a static sealing disk member compressed against the volumes disk member creating a gas seal and allowing for frictionless rotation there between;
a static base member comprising inlet ports and outlet ports for receiving compression fittings to fluidly connect the calibration valve to a gas chromatography system; and
an alignment ring member affixed to the base to generate a compressive force on the sealing disk member;
wherein the base member comprises an inlet port and an outlet port fluidly connected to a carrier gas, an inlet port and an outlet port fluidly connected to a measured gas used for calibration, and an inlet port and an outlet port fluidly connected to a sample chamber of the gas chromatograph; and
passing the sample comprising the measured gas through the gas chromatograph to detect and/or quantify the measured gas in the sample.

22. The method of claim 21, wherein the measured gas comprises hydrogen.

23. The method of claim 21, wherein the calibrating step facilitates the accurate detection of a volume of diffused hydrogen and the diffusion rate of small weld metal samples within a 5 mm³ to 100 mm³ range, following ISO standard 3690 for hydrogen measurement.
